(19) United States
(12) Patent Application Publication
(10) Pub. No.: US 2025/0262406 A1
(43) Pub. Date: Aug. 21, 2025

(54) FOLEY CATHETER SPONGE ASSEMBLY

(71) Applicant: Jenna Schaal, Bayville, NJ (US)

(72) Inventor: Jenna Schaal, Bayville, NJ (US)

(21) Appl. No.: 18/583,360

(22) Filed: Feb. 21, 2024

(51) Int. Cl. A61M 25/00; A61M 25/02

(52) U.S. Cl. CPC A61M 25/0017; A61M 25/02; A61M 2025/0253

(57) ABSTRACT

A foley catheter sponge assembly includes a foley catheter that has a tube and a balloon. A sponge is provided which has a longitudinal split integrated into the sponge thereby facilitating the tube of the foley catheter to be positioned in the sponge for securing the sponge on the tube. The sponge is strategically located with respect to the balloon to rest against a female patient's urethra when the tube is inserted into the female patient's urethra. In this way the balloon is secured against the female patient's bladder wall to inhibit the balloon from floating in the female patient's bladder. A pair of adhesive pads is each positioned in the longitudinal split to adhere to the tube of the foley catheter thereby securing the sponge to the tube.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to foley catheter devices and more particularly pertains to a new foley catheter device for securing a balloon of a foley catheter against a female patient's bladder wall. The device includes a cylindrical sponge that has a longitudinal cut such that the cylindrical sponge can be positioned around a tube of a foley catheter. The device includes a pair of adhesive pads that are each integrated into the cylindrical sponge which adhere to the tube for securing the cylindrical sponge at a strategic location on the tube such that the cylindrical sponge rests against the female patient's urethra to secure the balloon on the female patient's bladder wall.

### (2) Description of Related Art Including Information Disclosed Under 37 CFR 1.97 and 1.98

The prior art relates to foley catheter devices including a variety of urinary catheter devices which each at least includes a labial seal to rest against a female patient's labia for securing a catheter in place on a female patient and an external catheter device that has a disk shaped sponge which rests against a urethra of a patient and a urine catheter device that has a funnel integrated into a tube of the catheter for collecting urine that leaks from the patient's urethra. In no instance does the prior art disclose a foley catheter and a cylindrical sponge with a longitudinal cut to facilitate the cylindrical sponge to be positioned around a tube of the foley catheter which rests against a female patient's urethra to secure a balloon of the foley catheter against the female patient's bladder wall.

### BRIEF SUMMARY OF THE INVENTION

An embodiment of the disclosure meets the needs presented above by generally comprising a foley catheter that has a tube and a balloon. A sponge is provided which has a longitudinal split integrated into the sponge thereby facilitating the tube of the foley catheter to be positioned in the sponge for securing the sponge on the tube. The sponge is strategically located with respect to the balloon to rest against a female patient's urethra when the tube is inserted into the female patient's urethra. In this way the balloon is secured against the female patient's bladder wall to inhibit the balloon from floating in the female patient's bladder. A pair of adhesive pads is each positioned in the longitudinal split to adhere to the tube of the foley catheter thereby securing the sponge to the tube.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an exploded perspective view of a foley catheter sponge assembly according to an embodiment of the disclosure.

FIG. 2 is a top view of a sponge of an embodiment of the disclosure.

FIG. 3 is a front view of a sponge of an embodiment of the disclosure.

FIG. 4 is a cross sectional view taken along line 4-4 of FIG. 2 of an embodiment of the disclosure.

FIG. 5 is a perspective phantom view of a sponge of an embodiment of the disclosure.

FIG. 6 is a bottom perspective view of an embodiment of the disclosure showing a sponge being positioned around a tube of foley catheter.

FIG. 7 is a perspective in-use view of an embodiment of the disclosure.

### DETAILED DESCRIPTION OF THE INVENTION

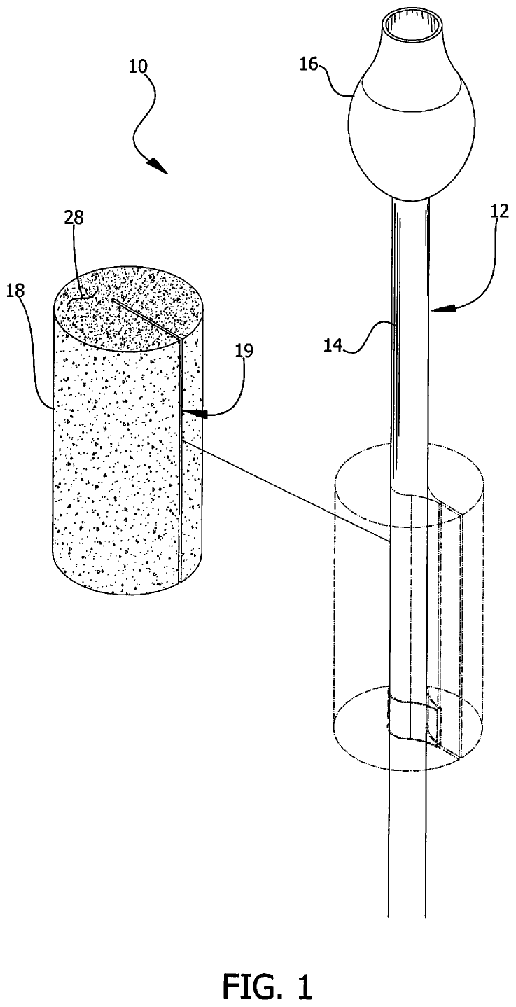

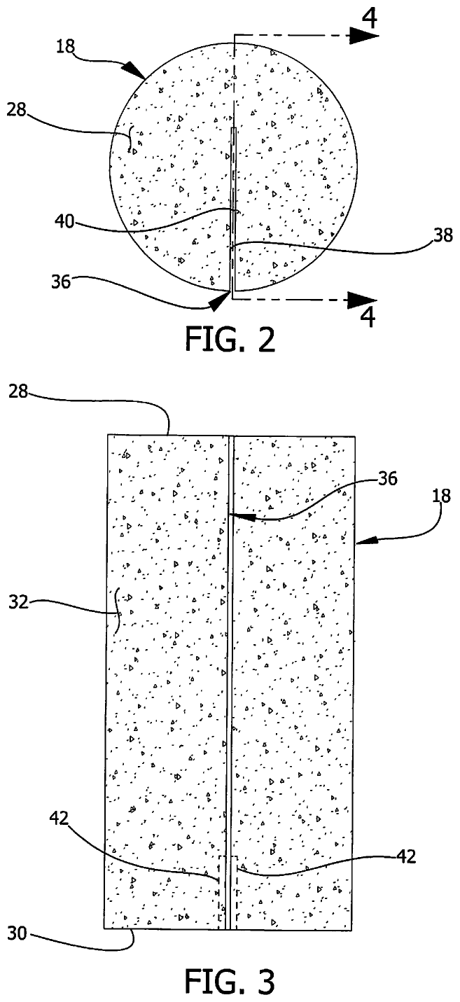

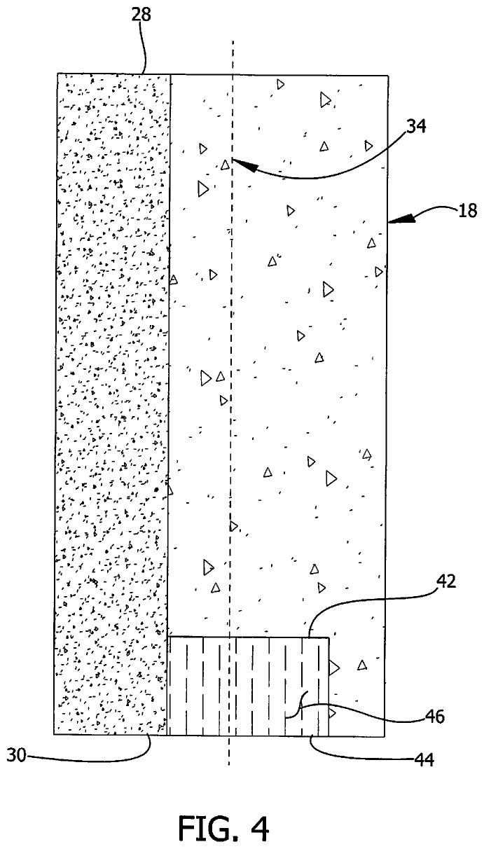

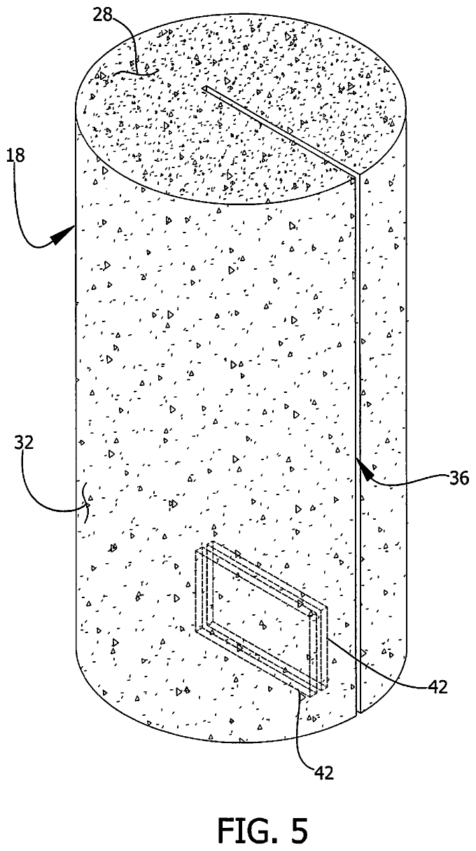

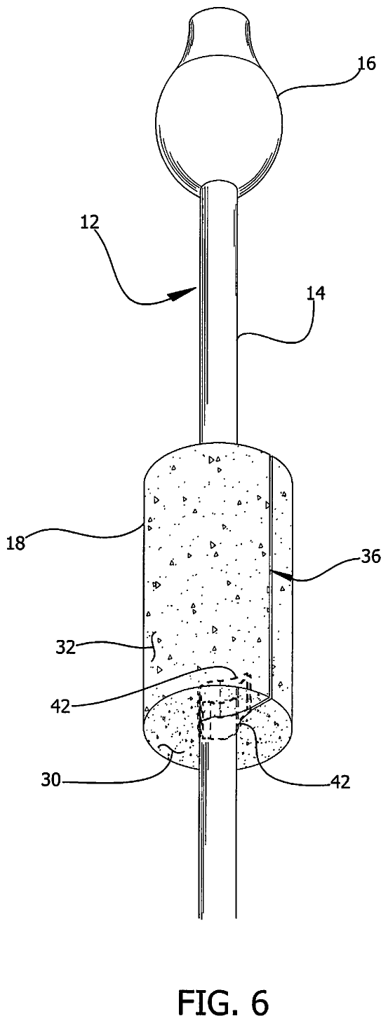

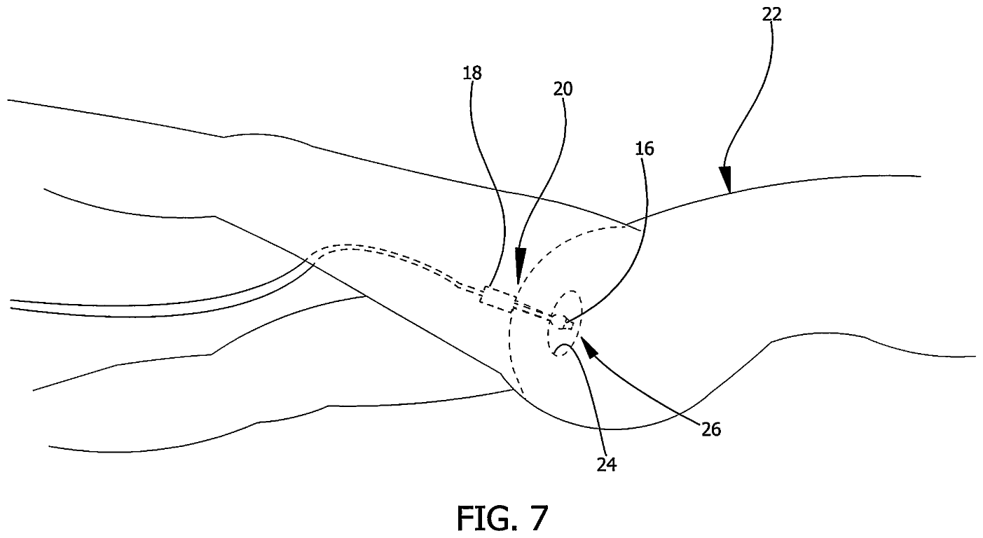

With reference now to the drawings, and in particular to FIGS. 1 through 7 thereof, a new foley catheter device embodying the principles and concepts of an embodiment of the disclosure and generally designated by the reference numeral 10 will be described.

As best illustrated in FIGS. 1 through 7, the foley catheter sponge assembly 10 generally comprises a foley catheter 12 that has a tube 14 and a balloon 16. A sponge 18 is provided which has a longitudinal split 19 integrated into the sponge 18 thereby facilitating the tube 14 of the foley catheter 12 to be positioned in the sponge 18 for securing the sponge 18 on the tube 14. The sponge 18 is strategically located with respect to the balloon 16 such that the sponge 18 rests against a urethra 20 of a female patient 22 when the tube 14 is inserted into the female patient's urethra 20. In this way the balloon 16 of the foley catheter 12 is secured against the wall 24 of the female patient's bladder 26 thereby inhibiting the balloon 16 from floating in the female patient's bladder 26. The sponge 18 is comprised of an antimicrobial material, including but not being limited to polyurethane foam or hemp palm, such that the sponge 18 inhibits the development of a urinary tract infection in the female patient 22. Furthermore, the sponge 18 is comprised of a fluid absorbent material to absorb urine that leaks from the female patient's urethra 20 thereby enhancing cleanliness for the female patient 22. The female patient 22 may be an elderly woman that suffers from incontinence or a surgical patient in recovery or any other female that is required to employ a foley catheter 12.

The sponge 18 has a top surface 28 and a bottom surface 30 and an outer surface 32 extending between the top surface 28 and the bottom surface 30 and the sponge 18 is elongated between the top surface 28 and the bottom surface 30. The outer surface 32 is continuously arcuate about a central longitudinal axis 34 of the sponge 18 such that the sponge 18 has a cylindrical shape. The sponge 18 has a cut 36 extending into the outer surface 32 and the cut 36 is elongated to extend through the top surface 28 and the bottom surface 30 such that the cut 36 defines the longitudinal split 19. Furthermore, the cut 36 is aligned with a central longitudinal axis 34 of the sponge 18. The cut 36 extends inwardly beyond the central longitudinal axis 34 of the sponge 18 such that the cut 36 has a depth that is greater than a radius of the sponge 18. In this way the tube 14 of the foley catheter 12 can be aligned with the central longitudinal axis 34 of the sponge 18 when the tube 14 is inserted into the cut 36. The cut 36 has a first bounding surface 38 and a second bounding surface 40 each extending between the top surface 28 and the bottom surface 30 of the sponge 18. and

A pair of adhesive pads 42 is provided and each of the pair of adhesive pads 42 is positioned in the longitudinal split 19. In this way each of the pair of adhesive pads 42 can adhere to the tube 14 of the foley catheter 12 when the tube 14 is inserted into the longitudinal split 19 thereby securing the sponge 18 to the tube 14. Thus, the pair of adhesive pads 42 inhibit the balloon 16 from displacing from the female patient's bladder wall 22. In this way the sponge 18 and the pair of adhesive pads 42 enhance the comfort of the female patient as well as inhibiting urine from leaking between the balloon 16 and the female patient's bladder wall 22.

Each of the pair of adhesive pads 42 is integrated into a respective one of the first bounding surface 38 and the second bounding surface 40 of the cut 36. The pair of adhesive pads 42 is aligned with each other and a lower edge 44 of each of the pair of adhesive pads 42 is aligned with the bottom surface 30 of the sponge 18. Each of the pair of adhesive pads 42 has an exposed surface 46 with respect to the respective first bounding surface 38 and the second bounding surface 40. Additionally, the exposed surface 46 of each of the pair of adhesive pads 42 adhesively engages the tube 14 when the tube 14 is positioned in the cut 36. Each of the adhesive pads 42 may comprise a fluid resistant and medical grade adhesive such that the adhesive pads 42 will not loosen from the tube 14 when the adhesive pads 42 are exposed to urine or other bodily fluids.

In use, the foley catheter 12 is inserted into the female patient's urethra 20 in compliance with standard medical practices such that the balloon 16 rests against the female patient's bladder wall 22. The sponge 18 is positioned around the tube 14 such that the sponge 18 rests against the female patient's urethra 20 and the pair of adhesive pads 42 is compressed against the tube 14 to adhere to the tube 14. In this way the sponge 18 inhibits the tube 14 from traveling further into the female patient's urethra 20 and displacing the balloon 16 from the female patient's bladder wall 22. Furthermore, the sponge 18 absorbs any urine that leaks from the female patient's urethra 20 thereby enhancing cleanliness and comfort for the female patient. Additionally, the sponge 18 can be removed and replaced on the tube 14 at any time when the sponge 18 becomes soaked with urine or for any other reason.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A foley catheter sponge assembly being positionable around a foley catheter tube at a strategic location to secure a balloon of the foley catheter against the bladder wall, said device comprising:
a foley catheter having a tube and a balloon;
a sponge having a longitudinal split integrated into said sponge thereby facilitating said tube of said foley catheter to be positioned in said sponge for securing said sponge on said tube, said sponge being strategically located with respect to said balloon wherein said sponge is configured to rest against a female patient's urethra when said tube is inserted into the female patient's urethra thereby securing said balloon against the female patient's bladder wall to inhibit said balloon from floating in the female patient's bladder, said sponge being comprised of an antimicrobial material wherein said sponge is configured to inhibit the development of a urinary tract infection in the female patient, said sponge being comprised of a fluid absorbent material wherein said sponge is configured to absorb urine that leaks from the female patient's urethra thereby enhancing cleanliness for the female patient; and
a pair of adhesive pads, each of said pair of adhesive pads being positioned in said longitudinal split thereby facilitating each of said pair of adhesive pads to adhere to said tube of said foley catheter when said tube is inserted into said longitudinal split thereby securing said sponge to said tube wherein said pair of adhesive pads is configured to inhibit said balloon from displacing from the female patient's bladder wall.

2. The assembly according to claim 1, wherein:
said sponge has a top surface and a bottom surface and an outer surface extending between said top surface and said bottom surface;
said sponge is elongated between said top surface and said bottom surface;
said outer surface is continuously arcuate about said central longitudinal axis of said sponge such that said sponge has a cylindrical shape;
said sponge has a cut extending into said outer surface;
said cut being elongated to extend through said top surface and said bottom surface such that said cut defines said longitudinal split; and
said cut is aligned with a central longitudinal axis of said sponge.

3. The assembly according to claim 2, wherein said cut extends inwardly beyond said central longitudinal axis of said sponge such that said cut has a depth being greater than a radius of said sponge thereby facilitating said tube of said foley catheter to be aligned with said central longitudinal axis of said sponge when said tube is inserted into said cut.

4. The assembly according to claim 2, wherein:
said cut has a first bounding surface and a second bounding surface each extending between said top surface and said bottom surface of said sponge; and
each of said pair of adhesive pads is integrated into a respective one of said first bounding surface and said second bounding surface of said cut; and
said pair of adhesive pads is aligned with each other.

5. The assembly according to claim 4, wherein:
a lower edge of each of said pair of adhesive pads is aligned with said bottom surface of said sponge;
each of said pair of adhesive pads has an exposed surface with respect to said respective first bounding surface and said second bounding surface; and
said exposed surface of each of said pair of adhesive pads adhesively engages said tube when said tube is positioned in said cut.

6. A foley catheter sponge assembly being positionable around a foley catheter tube at a strategic location to secure a balloon of the foley catheter against the bladder wall, said device comprising:
a foley catheter having a tube and a balloon;
a sponge having a longitudinal split integrated into said sponge thereby facilitating said tube of said foley catheter to be positioned in said sponge for securing said sponge on said tube, said sponge being strategically located with respect to said balloon wherein said sponge is configured to rest against a female patient's urethra when said tube is inserted into the female patient's urethra thereby securing said balloon against the female patient's bladder wall to inhibit said balloon from floating in the female patient's bladder, said sponge being comprised of an antimicrobial material wherein said sponge is configured to inhibit the development of a urinary tract infection in the female patient, said sponge being comprised of a fluid absorbent material wherein said sponge is configured to absorb urine that leaks from the female patient's urethra thereby enhancing cleanliness for the female patient, said sponge having a top surface and a bottom surface and an outer surface extending between said top surface and said bottom surface, said sponge being elongated between said top surface and said bottom surface, said outer surface being continuously arcuate about said central longitudinal axis of said sponge such that said sponge has a cylindrical shape, said sponge having a cut extending into said outer surface, said cut being elongated to extend through said top surface and said bottom surface such that said cut defines said longitudinal split, said cut being aligned with a central longitudinal axis of said sponge, said cut extending inwardly beyond said central longitudinal axis of said sponge such that said cut has a depth being greater than a radius of said sponge thereby facilitating said tube of said foley catheter to be aligned with said central longitudinal axis of said sponge when said tube is inserted into said cut, said cut having a first bounding surface and a second bounding surface each extending between said top surface and said bottom surface of said sponge; and
a pair of adhesive pads, each of said pair of adhesive pads being positioned in said longitudinal split thereby facilitating each of said pair of adhesive pads to adhere to said tube of said foley catheter when said tube is inserted into said longitudinal split thereby securing said sponge to said tube wherein said pair of adhesive pads is configured to inhibit said balloon from displacing from the female patient's bladder wall, each of said pair of adhesive pads being integrated into a respective one of said first bounding surface and said second bounding surface of said cut, said pair of adhesive pads being aligned with each other, a lower edge of each of said pair of adhesive pads being aligned with said bottom surface of said sponge, each of said pair of adhesive pads having an exposed surface with respect to said respective first bounding surface and said second bounding surface, said exposed surface of each of said pair of adhesive pads adhesively engaging said tube when said tube is positioned in said cut.

7. A method of securing a foley catheter in a female patient, the steps of the method comprising:
inserting a foley catheter into a female patient's urethra in a manner that complies with standard medical practices;
positioning a sponge around a tube of said foley catheter, said sponge having a cut which extends through an outer surface of said sponge and is elongated to extend through each of a top surface and a bottom surface of said sponge, said cut insertably receiving said tube of said foley catheter such that said sponge extends along a substantial length of said tube;
positioning said sponge at a point along said tube such that said sponge rests against the female patient's urethra thereby securing a balloon of said foley catheter against a wall of the female patient's bladder thereby inhibiting said balloon from floating in the female patient's bladder; and
compressing a pair of adhesive pads against said tube, each of said pair of adhesive pads being positioned in said cut in said sponge against said tube such that said pair of adhesive pads adhesively engages said tube thereby securing said sponge on said tube.
